(19) United States
(12) Patent Application Publication
(10) Pub. No.: US 2003/0073963 A1
(43) Pub. Date: Apr. 17, 2003

(54) Bowel irrigation system and probe therefor

(76) Inventor: Malcolm Ian Falconer, London (GB)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 10/058,347

(22) Filed: Jan. 30, 2002

(30) Foreign Application Priority Data
Oct. 12, 2001 (DK) PA 2001 01509

(51) Int. Cl. A61M 1/00

(52) U.S. Cl. 604/328; 604/322

(57) ABSTRACT

A bowel irrigation system having a probe connected to a reservoir that contains irrigating liquid. The reservoir is a sealed container acted upon by a pumping element. The user operates the pumping element to create pressure on the sealed container and thereby displace the liquid from the reservoir into the probe. An inflatable cuff may also be included for securing the position of the probe when in use. The system is portable and may be employed by a user without outside assistance.

### FIELD OF THE INVENTION

This invention relates to a bowel irrigation system and more particularly but not exclusively to a portable system designed for use by disabled persons with limited limb movement for instance due to a spinal cord injury or being spina bifida or MS sufferers. The invention also relates to an irrigation probe for use with such a system.

### BACKGROUND OF INVENTION

Due to the disabilities suffered by the aforementioned groups of persons, it is quite common that they cannot perform their normal bowel function without some form of outside help. This is usually in the form of drugs, digital stimulation, massage or colonic irrigation. Currently available colonic irrigation kits can be awkward to use and almost exclusively have to be used with the help of a third party.

Various bowel irrigation systems are known. One such system is the gravity fed arrangement which is disclosed in our published International application No. WO98123312 in which the irrigation medium is fed by gravity from a bag hung from a frame arranged close to the patient. Flow of liquid from the bag to the user is controlled by a flow regulation device operated by the user. The problem with this system is that it relies on gravity feed so the bag needs to be located above the user in some way and it is therefore usually suspended from a frame. This means that the system cannot be readily portable unless the frame can be collapsed for storage. Portability of equipment is extremely important to disabled persons who are not hospitalised or bed-ridden if they are to lead as normal a life as possible and deal with their daily bodily functions. This is particularly important if they travel away from their home, for instance, to someone else's home or if they stay in a hotel. In this situation, they need to be able to deal with their bowel function easily and equipment currently available does not allow them to do this. Systems have been tried where the bag containing the irrigation liquid can be mounted on the wall of a bathroom using suction or adhesive pads but such systems have been found not to work particularly well because quite often the pads become detached from the wall and the bag falls to the floor spilling the contents all over it.

Bowel care systems are known which are movable on wheels such as those disclosed in U.S. Pat. Nos. 5,019,056 or 4,874,363. These systems comprise a reservoir for the liquid irrigation fluid, a tank for discharged material, a probe for insertion into the patient and ancillary equipment including a pump to pump the irrigation liquid from the reservoir through the pump to the probe. The system is mounted on a carriage with wheels so that it can be moved from one location to another. However, although such systems are mobile, they are heavy and cumbersome and they are therefore not readily transportable by the user. They therefore tend to be restricted to use in hospitals or nursing homes where they can be wheeled from one patient to another. Because of their size and weight, these systems cannot be carried by their users from one place to another and thereby allow them to lead as normal a life as possible.

### BRIEF DESCRIPTION OF INVENTION

It is therefore a first object of the invention to provide a bowel irrigation system which can be readily carried or transported by the user from one place to another during their daily lives so that they can lead as normal a life as possible.

It is another object of the invention to provide a bowel irrigation system which is easy to use and avoids the need for third party assistance to carry out the bowel management function and thereby provide the user with a greater degree of freedom to attend to their own bowel motion at any time.

It is a still further object of the invention to provide a probe which is easier to use than prior art probes.

According to a first aspect of the invention, there is provided a bowel irrigation system comprising a reservoir to contain an irrigation liquid with a probe connected thereto and means for transferring irrigation liquid from the reservoir to the probe, the invention being characterised in that the reservoir is a sealed container and the pumping means are operable by the user to create a pressure in the container to displace the liquid therefrom and feed it to the probe.

Preferably the pumping means is a compressible member such as a resilient bulb or bellows connected to the reservoir so that, in use, compression of the bulb pressurises the liquid in the reservoir and displaces it and feeds it to the probe. The bulb or bellows can either be manually operated by the user or, in certain circumstances, it may be more convenient for the user to place the bulb or bellows elsewhere e.g. under the chin or under their armpit and compress it by moving their chin or arm towards and away from their body. The pumping means could however be provided by compressed gas, by gravity or by a pump powered by electricity (mains or low voltage), by gas or by hydraulic fluid.

In order to render the irrigation system as portable as possible, the reservoir preferably has a capacity of less than 5 litres. If however the system is to be used for repeated irrigation, a larger capacity reservoir may be necessary.

Further, in order to minimise the size of the system during transportation, the container can be used for storing probes and tubes etc. thereby making transportation easier for the user.

In the preferred embodiment, the compressible member and probe are connected to the reservoir by a flexible tube.

Conveniently the probe has a connector at one end thereof to releasably connect it to the flexible tube connecting it to the reservoir.

Another problem associated with prior art anal irrigation systems concerns the difficulty the user has in inserting the probe and correctly positioning it in the anal opening. U.S. Pat. No. 3,459,175 discloses an example of a prior art probe which has a shaft through which water can be supplied to the colon. The probe has an abutment around it which contacts the user's posterior on insertion of the probe in the anal opening and thereby limits the extent to which the probe can penetrate it. Spaced from this abutment is a balloon which is connected by a flexible tube to a syringe. Activation of the syringe forces air into the balloon to inflate it and thereby axially locate the probe in the anal opening and prevent its removal therefrom until the balloon is deflated. Having to inflate the balloon using a separate syringe complicates bowel management considerably for the user so such systems are extremely difficult to operate without outside assistance.

It is therefore a further object of the invention to provide a probe which is easy for the user to insert and remove without the need for outside assistance or easy to use if the probe has to be administered by a carer or helper if the user is unable to handle it alone.

According to a further aspect of the invention therefore, there is provided a probe for use in a bowel irrigation system comprising a shaft having a proximal end and a distal end and a duct therethrough for supplying irrigation liquid from the proximal end to an outlet at the distal end, an inflatable cuff mounted on the shaft adjacent said outlet to retain, in use, the probe in position in the user's anal opening, the invention being characterised by manually operable cuff inflation means operable to inflate the cuff, said cuff inflation means having associated therewith a one-way valve to admit ambient air to the interior thereof only during inflation of the cuff and means operable to release pressurised air from within the inflated cuff to deflate the said cuff.

The inflatable cuff is in a preferred embodiment mounted on the shaft of the probe adjacent its proximal end. Compared to prior art probes it simplifies handling and makes use of the probe easier to the user.

Preferably the manually operable cuff inflation means is a resilient compressible bulb mounted on the shaft of the probe. As an alternative, however, the cuff inflation means and the pumping means can be provided by one and the same unit, further including means for directing the pressure either to the cuff or to the reservoir.

In the preferred embodiment, the shaft has an outlet opening into the interior of the cuff, a separate duct connecting the interior of the bulb with said outlet.

Conveniently the pressure release means comprises a movable member operable to open the one-way valve to vent the contents of the inflated cuff to atmosphere.

In the preferred embodiment, the movable member is spring biased to a normal closed position, movement of said member opening the valve to release the pressure within the bulb and deflate the cuff.

Conveniently the movable member is a depressible button which forms part of the one-way valve. In the preferred embodiment, the one-way valve is located on the bulb and the button has a finger protruding therefrom which cooperates with the one-way valve to open it when the button is depressed.

According to a third aspect of the invention there is provided a probe for use in a bowel irrigation system in which irrigation liquid is supplied to an outlet adjacent the end of the probe, the probe comprising a shaft having a proximal end and a distal end and a duct therethrough for the supply of said irrigation liquid to the outlet, and an inflatable cuff mounted on the shaft, characterised in that the probe end is detachable and includes separate fluid supply means on the shaft to supply fluid to the interior of the cuff, the proximal end of the shaft being formed to make a releasable connection with the fluid supply used to inflate the cuff and the supply of irrigation liquid. The advantage of making the probe end readily releasable from the cuff inflation fluid source and the irrigation liquid source is that it can be made as a replaceable item which can be used once and then thrown away. This is particularly important as it means that a sterilised probe can be supplied to the user in a sealed sachet and disposed of after use thus rendering it very hygienic to use.

### BRIEF DESCRIPTION OF THE DRAWINGS

The invention will now be described, by way of example only, with reference to the accompanying drawings, in which:

FIG. 1 is a perspective view of a portable bowel irrigation system of the invention;

FIG. 2 is a schematic view showing the system of FIG. 1 in use;

FIG. 3 is a side view of one form of probe for use with the system shown in FIGS. 1 and 2;

FIG. 4 is an enlarged cross section of the probe shown in FIG. 3;

FIG. 4A is a view of the probe shown in FIGS. 3 and 4 but with the replaceable probe end separated from the remainder of the probe;

FIG. 5 is a view showing the probe of FIGS. 3 and 4 in position in a user; and

FIG. 6 is an enlarged cross section of the valve shown in FIGS. 3-5.

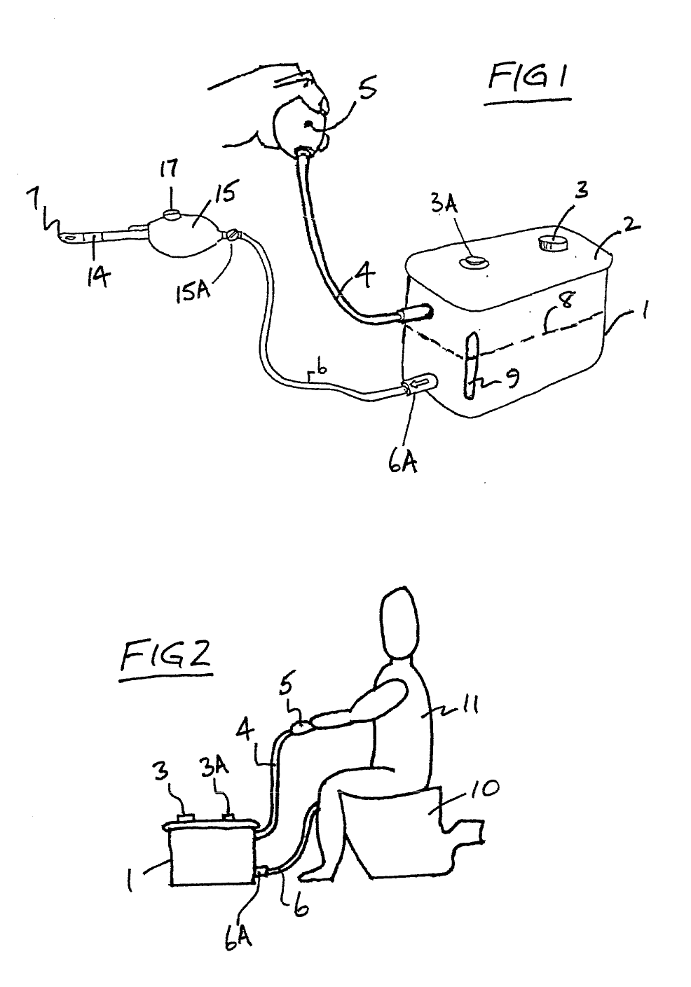

Referring now to the drawings, there is shown in FIG. 1 a bowel irrigation system which comprises a free standing reservoir 1 for the irrigation liquid with a top 2 having an inlet therein closed by a screw cap 3. The top 2 makes an airtight seal with the reservoir 1 to provide a sealed container for the irrigation liquid. A pressure relief valve 3A is mounted on the lid 2 for reasons to be explained hereafter. The side wall of the reservoir 1 can have an optional sight glass 9 in it to indicate the level of liquid 8 inside the reservoir 1.

A flexible tube 4 is connected to the side wall of the reservoir 1 and the outlet therefrom opens into the interior of the reservoir 1 above the upper level 8 of the liquid therein. A compressible member in the form of a rubber bulb 5 is attached to the end of the tube 4.

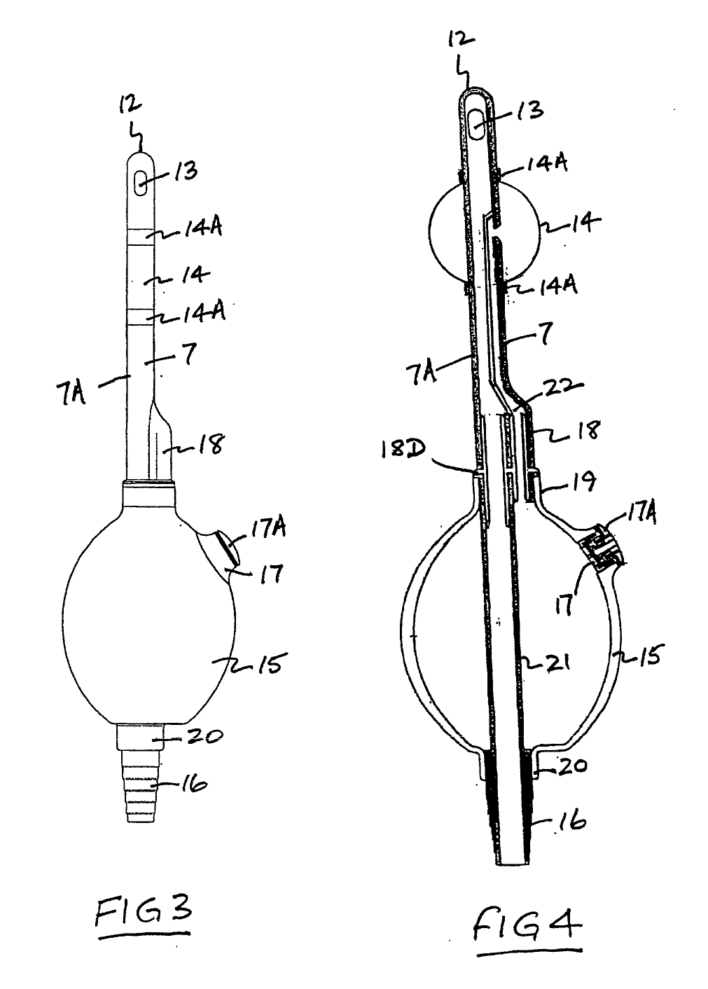

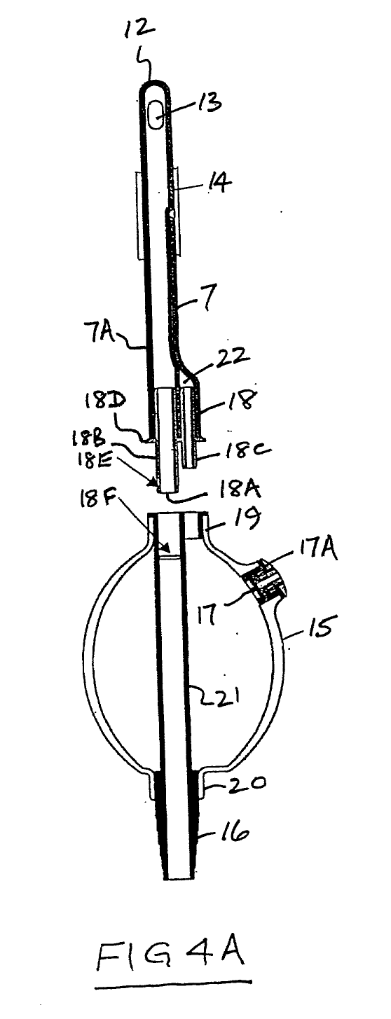

A second tube 6 is also connected at one end to the reservoir 1 via a one-way valve 6A which opens into the bottom thereof below the liquid level 8. A probe 7 with manually operable cuff inflation means in the form of compressible bulb 15 is attached to the free end of the flexible tube 6. The probe 7, preferably provided with a relatively small diameter to ease insertion of the probe into the body opening, will be described hereafter in more detail with reference to FIGS. 3 and 4.

The probe 7 comprises a detachable end shaft 7A having a distal end 12 with a liquid outlet 13 therein. In connection with this invention, the distal end of the probe is understood to be the insertable end of the probe. The end shaft 7A also has a second opening 23 therein which communicates with the interior of an inflatable cuff 14 whose edges 14A are secured in an airtight manner to the shaft 7A in known manner e.g. using an adhesive, a heat weld or a thread. A secondary duct 22 is provided within the body of the probe and communicates with the interior of the cuff 14 via outlet 23.

A moulded plastic connector 18A is fitted in the proximal end of the probe 7 and releasably connects the detachable end 7A of the probe to the compressible bulb 15 as can be seen in FIG. 4A. This allows the probe to be separated from the bulb 15 after each irrigation and replaced with a new one.

The connector 18A comprises a first tube 18B and a second shorter tube 18C with an annular shoulder 18D intermediate its ends which seats against the proximal end of the detachable probe end 7A. An annular rib 18E is formed around one end of tube 18B which locates in an annular groove 18F around the interior of the tube 21. It will be appreciated therefore that when the probe 7 is pushed into the bulb 15, the tubes 18B and 18C slide into the bulb 15 until the annular shoulder 18D seats against the bulb 15, and the rib 18E on the connector tube 18B locates in the groove 18F in the tube 21 to retain it therein. As the connector 18A makes a snap connection with the tube 21, it can be readily separated therefrom by applying an axial force thereto.

A liquid supply tube 21 extends through the compressible bulb 15 and has a connector 16 at its proximal end to which liquid supply tube 6 can be attached. The compressible bulb 15 is attached to the main feed tube 21 in an airtight manner by securing annular flanges 19 and 20 thereto in known manner, e.g. using an adhesive, a heat weld or thread. A tap or clamping tube 15A can be provided in the tube 6 between the reservoir 1 and the probe 7 (see FIG. 1). This can either be a separate tap or incorporated into connector 16 of probe 7 (see FIGS. 3 and 4).

Referring now to FIGS. 3, 4 and 6, a valve assembly 17 having a depressible button 17A is mounted in the wall of the bulb 15. The valve assembly 17 has a flap or flutter valve 26 therein (see FIG. 6) which is axially movable in recess 27A and normally seals against seat 27 in its rest position. However, when the bulb 15 is compressed, air is sucked into the interior of the bulb 15 through the valve 17 which lifts the flap valve 26 off its seat 27. On release of the compressive force on the bulb 15, air pressure in the bulb 15 forces the flutter valve 26 to reseat on the seat 27 and form a seal therewith. Thus, the valve 17 operates as a one-way valve which only allows ambient air to enter the bulb 15 when it is compressed. The pressurised air in the bulb 15 then travels via secondary inner duct 22 to the interior of the cuff 14 via outlet 23. As a result, the cuff 14 is inflated from its deflated condition shown in FIG. 3 to its inflated condition shown in FIG. 4.

To deflate the cuff 14, the user presses on the button 17A which compresses resilient legs 17C and allows the pin 17B to come into contact with the flutter valve 26 and push it away from sealing engagement with the seat 27. As a result, the pressurised air within the inflated cuff 14 and the bulb 15 can vent to atmosphere so the cuff 14 deflates and returns to its configuration shown in FIG. 3. Release of the pressure on the button 17A allows the flutter valve 26 to reseat on the seat 27 and form a seal therewith.

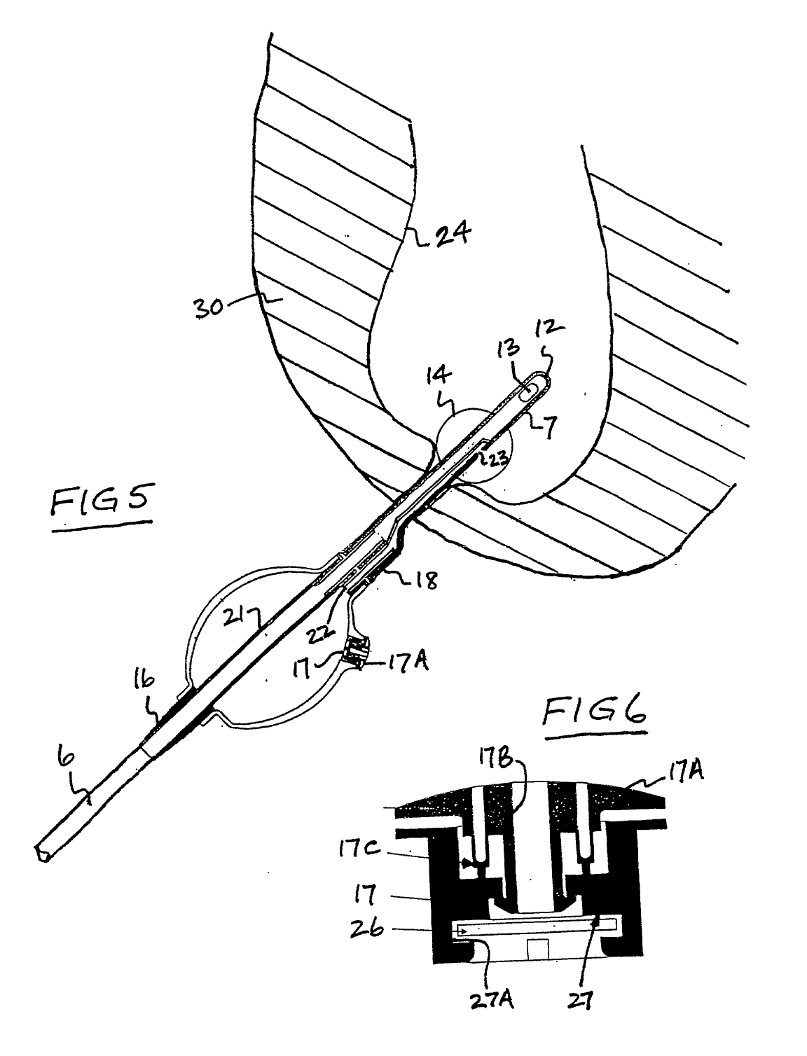

FIG. 5 shows the probe 7 of FIGS. 3 and 4 in position in anal opening 25 of a user. It can be seen that the enlarged portion 18 of the probe 7 limits the axial extent to which the probe can be inserted into the anal opening 25. When the cuff 14 is inflated, it seats on the entry of the anal opening into the colon 24 and axially locates the probe 7 therein.

One way in which the bowel irrigation system of the invention is used by a user, carer or helper will now be described with reference to FIG. 2.

As can be seen from the drawing, user 11 sits on toilet 10. If the user is in a wheelchair, it will be appreciated that this should not be too difficult as it is possible to slide from the wheelchair onto the toilet. The liquid reservoir 1 is placed on the floor opposite the user and the user 11 can then insert the tube 6 with probe 7 at its distal end into position as shown in FIG. 5. The system is primed by pumping water to the probe prior to insertion of the probe into position in order to avoid introducing air into the bowel system of the user. The probe 7 of the present invention makes this procedure much easier because the user can grip the bulb 15 quite readily and push the probe 7 into the anal opening 25 until the bulb 5 abuts the outer surface 29 of the user's posterior 30. Once this occurs, the user knows that the probe 7 is fully inserted. The user can then start squeezing the bulb 15 to inflate the cuff 14 to axially locate the probe 7 in the anal opening 25 as shown in FIG. 5. This is an operation which the user can easily perform as the user is already holding the bulb 15. It is therefore very easy for the user to simply squeeze the bulb and inflate the cuff 14.

Once the cuff 14 has been inflated and the probe 7 is properly located in the anal opening 25, the user can then repeatedly squeeze the other bulb 5 to pressurise the container 1 and thereby displace the liquid therein through the duct 6 to the probe 7 where it exits via outlet 13 into the colon 24. So long as the user continues to squeeze the bulb 5, the liquid will continue to flow. It stops when the user stops squeezing the bulb 5. Pressure relief valve 3A prevents over-pressurisation of the container and thus the pressure of the water entering the bowel. The valve 3A is preferably set to vent at approximately 2.5 m of water to avoid the risk of exposing the bowel system to a harmful pressure.

The valve 3A can be a simple pressure relief valve of known type which releases at the pre-set pressure or it could be a simple stopper which pops out of the lid at the required predetermined pressure. As a consequence thereof the valve 3A can easily be manually operated to release the pressure built up in the container when the user for one reason or another wishes to stop administration of the irrigation liquid.

Once sufficient irrigating liquid has been pumped into the colon, the user presses the button 17A to vent the air in the inflated cuff 14 to atmosphere. The cuff therefore collapses back to its original configuration shown in FIG. 3 which allows the user to withdraw the probe from the anal opening 25. Once this has been done, water and faeces in the colon can leave the anal opening for collection in the toilet 10.

The one-way valve 6A prevents the possibility of back flow of contaminated liquid into the reservoir and the tap or clamp 15A prevents the flow of water to the probe 7 should the probe outlet 13 be positioned below the water level in the reservoir 1 at any time.

It will be seen from the foregoing description that the bowel irrigation system of the present invention provides a very simple and compact apparatus which is easy to use so a user's bowel management is dramatically simplified. It also is readily transportable from place to place thereby giving the patient greater freedom to travel away from home without the need to have a third party assist them with their bowel management.

Although the invention has been described in relation to its use in a retrograde irrigation procedure, i.e. one in which the probe is inserted into the user's anal opening, it could be used in any irrigation procedure be it e.g. a stomal, ileostomal or an antegrade irrigation procedure whereby the probe is inserted into the exposed end of a stoma protruding through the user's stomach wall and connected to the user's bowel.

The irrigation medium to be used with the system of the invention can be any suitable medium e.g. tap water, isotonic salt water, sterile water or oily substances.

Preferred materials for the bulb 5 and the cuff inflation bulb 15 can be any suitable material e.g. such as PVC, TPE or PU.

Preferred materials for the inflatable cuff 14 are PU, latex or PVC although other materials providing similar properties can likewise be used.

1. A bowel irrigation system comprising a reservoir (1) for irrigating liquid with a probe (7) connected thereto and means for transferring the irrigating liquid from the reservoir to the probe characterised in that the reservoir (1) is a sealed container and the pumping means (5) is operable by the user to create a pressure in the container (1) to displace the liquid therefrom and feed it to the probe (7).

2. A system as claimed in claim 1 characterised in that the pumping means is a compressible member (5) e.g. a resilient bulb or bellows which is connected to the reservoir (1) so that, in use, applying a compressive force to the bulb (5) pressurises the liquid in the reservoir and feeds said liquid to the probe (7).

3. A system as claimed in claim 2 characterised in that the compressible member (5) is connected to the reservoir (1) by a flexible tube (4) and the probe (7) is connected to the reservoir (1) by a flexible tube (6) which incorporates a one-way valve which allows liquid to flow only from the reservoir to the probe.

4. A system as claimed in claim 3 characterised in that a tap or clamp (15A) is provided between the reservoir (1) and the probe (7).

5. A probe for use in a bowel irrigation system comprising a shaft having a proximal end and a distal end and a duct therethrough for supplying irrigation liquid from the proximal end to an outlet at the distal end, an inflatable cuff mounted on the shaft adjacent said outlet to retain, in use, the probe in position in the user's anal opening characterised by manually operable cuff inflation means (15) operable by the user to inflate the cuff (14), said cuff inflation means (15) having associated therewith a one-way valve (17) to admit ambient air to the interior thereof only during inflation of the cuff (14) and retain said air therein and means (17A,17B) operable to release the pressure within the inflated cuff to deflate said cuff.

6. A probe as claimed in claim 5 characterised in that the manually operable cuff inflation means is a resilient compressible bulb (15) mounted on the shaft (7).

7. A probe as claimed in claim 6 characterised in that the shaft (7) has an outlet (23) opening into the interior of the cuff (14), a separate duct (22) connecting the interior of the bulb (15) with said outlet (23).

8. A probe as claimed in claim 6 or claim 7 characterised in that the pressure release means comprises a movable member (17A) normally biased to a closed position, movement of said member opening the valve (17) to vent the contents of the cuff (14) to atmosphere and deflate the cuff (14).

9. A probe as claimed in any of claims 6-8 characterised in that the movable member (17A) is a depressible button which forms part of the one-way valve (17).

10. A probe as claimed in claim 9 characterised in that the one-way valve (17) is located on the bulb (15).

11. A probe as claimed in any of claims 5-10 characterised in that the cuff inflation means (15) includes a connector (18A) operable to releasably connect the probe shaft (7) thereto.

12. A probe for use in a bowel irrigation system in which irrigation liquid is supplied to an outlet adjacent the end of the probe, the probe comprising a shaft having a proximal end and a distal end and a duct therethrough for the supply of said irrigation liquid to the outlet, and an inflatable cuff mounted on the shaft characterised in that the probe end is detachable and includes separate fluid supply means (22) on the shaft (7A) to supply a fluid to the interior of the cuff (14), the proximal end of the shaft (7A) being formed to make a releasable connection with the fluid supply used to inflate the cuff and the supply of irrigation liquid.

13. A probe as claimed in claim 12 characterised in that the secondary fluid supply means is a secondary duct (22) inside the probe shaft (7A).
